(19) United States
(12) Patent Application Publication
(10) Pub. No.: US 2013/0345671 A1
(43) Pub. Date: Dec. 26, 2013

(54) MICRONEEDLE-CONTAINING DRUG DELIVERY DEVICE TO BE ATTACHED TO EXTERIOR WALL OF VASCULAR VESSEL AND METHOD FOR DRUG DELIVERY THEREWITH

(71) Applicants: Won Hyoung Ryu, Goyang-si (KR); Jin Bum Kim, Changwon-si (KR); Chang Kuk Choi, Daegu (KR); Young Nam Youn, Seoul (KR)

(72) Inventors: Won Hyoung Ryu, Goyang-si (KR); Jin Bum Kim, Changwon-si (KR); Chang Kuk Choi, Daegu (KR); Young Nam Youn, Seoul (KR)

(73) Assignee: INDUSTRY-ACADEMIC COOPERATION FOUNDATION YONSEI UNIVERSITY, Seoul (KR)

(21) Appl. No.: 14/002,771

(22) PCT Filed: Sep. 30, 2011
(86) PCT No.: PCT/KR11/07219

§ 371 Date: Sep. 3, 2013

(30) Foreign Application Priority Data
Mar. 4, 2011 (KR) 10-2011-0019497

(51) Int. Cl. A61M 37/00

(52) U.S. Cl. CPC A61M 37/0015
USPC 604/506; 604/173; 604/257

(57) ABSTRACT

A drug delivery device, which is to be attached to the exterior wall of a vascular vessel, includes: a) a biocompatible device body for surrounding a vascular vessel; b) one or plurality of biocompatible microneedle, which are connected to the interior of the biocompatible device body and capable of penetrating up to the tunica media so as to deliver drug to the vascular smooth muscle cells. The drug delivery device and method enable spatial and temporal control of the drug ejected onto the blood cells and enable insertions of two or more different drugs at different delivery speeds and durations, thereby significantly improving the treatment success rate.

### TECHNICAL FIELD

The present invention relates to a drug delivery device for attachment to the outer wall of a blood vessel, which comprises a microneedle, and more particularly to a drug delivery device for attachment to the outer wall of a blood vessel, which comprises: a) a body made of a biocompatible material and formed so as to cover the blood vessel; and b) one or more needles made of a biocompatible material, which are connected to the inside of the body and inserted into the tunica media of the blood vessel so as to deliver a drug to vascular smooth muscle cells, and to a method for delivering a drug using the same. When the drug delivery device and drug delivery method of the present invention are used, a drug can be released into vascular cells in a spatially and temporally controlled manner, and two or more different drugs can be delivered at different delivery rates during different periods, and thus the success rate of treatment can be significantly increased.

### BACKGROUND ART

Stents are used to treat vascular stenosis caused by cardiac atherosclerosis or peripheral vascular atherosclerosis, or bypass surgery using venous or arterial grafts is used to treat restenosis following stent surgery.

Due to the stenosis or occlusion of grafts, 30-50% of patients within 5 years after the surgery and 50% or more of patients within 10 years after the surgery require re-surgery or lead to death.

It is known that the stenosis or occlusion of vascular grafts is caused by hyperplasia (vascular constriction) resulting from the abnormal growth of vascular smooth muscle cells due to damage to endothelial cells, which occur during surgery.

It is known that the administration of drugs for controlling the abnormal growth of vascular smooth muscle cells and drugs for promoting the re-growth of endothelial cells is most effective for preventing the stenosis or occlusion of vascular grafts. However, a method capable of delivering the optimum amounts of such drugs to a desired site during a desired period has not yet been reported.

Methods developed top date include a method in which a gel-type structure as shown in FIGS. 1a and 1b is attached to the outer wall of blood vessels so that the drug in the gel is delivered due to the expansion of the gel. This structure is easily attached and conveniently operated, but has problems in that the drug is difficult to deliver sufficiently from the outside of blood vessels to the tunica media (target location) of blood vessels and is highly likely to be scattered, and thus only a small amount of the total amount of the drug is delivered into the vascular tissue.

Another method is a method in which branched blood vessels are covered with a cuff-type structure made of a polymer material as shown in FIG. 2. In this method, a drug is attached or applied to the inside of the cuff so that it comes into contact with the outer wall of blood vessels and is delivered into the blood vessels. However, this method has problems in that the drug is difficult to deliver from the outer wall into blood vessels and in that surface friction caused by the constriction and dilation of blood vessels make it possible to control the delivery period of the drug.

In addition, a method that uses gene replacement and augmentation by viral delivery was developed. In this method, a gene is delivered to vascular tissue cells using adenovirus or lentivirus in order to control the growth and differentiation of cells. It was shown that this method can affect the abnormal growth of cells in the tunica intima of blood vessels, but it was reported that the effect of this method is significantly low (X. Yang et al, Circulation 104, 2001, 1588-1590).

In addition, a method of controlling the abnormal growth of vascular cells using gene inhibition was also studied. In this method, drugs of oligonucleotides, anti-sense oligonucleotides or transcription factor decoys are used to arrest the cell cycle and prevent the abnormal growth of vascular cells, and small interfering RNA is used to control a portion or the cell cycle (M. S. Conte, J. Vasc. Surg. 45, 2007, 74A-81A).

It is known that this method can control the abnormal growth of vascular cells using a specific gene, but the effect of use thereof has not yet been verified due to the absence of a method capable of delivering the drug to a desired location during a predetermined period.

In an attempt to overcome such problems and to improve therapeutic effects by local drug delivery, a method was recently reported in which E2F (edifoligide) that is an oligodeoxynucleotide decoy is adsorbed into a venous graft immediately before transplantation to prevent the abnormal growth of vascular smooth muscle cells. It was confirmed in animal tests and clinical trials that this method has improved effects compared to existing methods (M. S. Conte et al, Vasc. Endovascular Surg. 39, 2005, 15-23). However, there is an urgent need to develop a drug delivery method that can be more precisely temporally and spatially controlled.

### DISCLOSURE

### Technical Problem

The present invention has been made in order to solve the above-described problems, and it is an object of the present invention to provide a drug delivery device comprising a microneedle, which can be attached to the outer wall of a blood vessel, can exactly deliver a desired amount of a drug to the vascular tunica media having vascular smooth muscle cells (target location) during a predetermined period and does not require separate removal surgery, as well as a drug delivery method.

Another object of the present invention is to provide a drug delivery device for attachment to the outer wall of a blood vessel, in which the drug delivery device can be specifically made according to the location of a transplanted blood vessel and cells and is fixed to the outer wall of the blood vessel through the needle so that the detachment of the device by the constriction and dilation of the blood vessel, as well as a drug delivery method.

Still another object of the present invention is to provide a drug delivery device for attachment to the outer wall of a blood vessel and a drug delivery method, which can deliver two or more different drugs at different delivery rates during different periods and achieve various delivery profiles to significantly increase the success rate of treatment.

### Technical Solution

In order to accomplish the above objects, the present invention provides a drug delivery device for attachment to the outer wall of a blood vessel, the blood delivery device comprising: a) a body made of a biocompatible material and formed so as to cover the blood vessel; b) one or more needles made of a biocompatible material, which are connected to the inside of the body and inserted into the tunica media of the blood vessel so as to deliver a drug to vascular smooth muscle cells; c) one or more drug reservoirs formed in the body; and d) microchannels formed in the needles and serving to deliver the drug from the drug reservoirs to the tunica media of the blood vessel. Herein, the rate and period of drug delivery by diffusion and osmotic pressure may be controlled by controlling the structure of the microchannels, and the control of the drug delivery rate and period may be achieved by controlling the length, cross-sectional area or shape of the channels.

In another embodiment, the present invention provides a drug delivery device for attachment to the outer wall of a blood vessel, the blood delivery device comprising: a) a body made of a biocompatible material and formed so as to cover the blood vessel; b) one or more needles made of a biocompatible material, which are connected to the inside of the body and inserted into the tunica media of the blood vessel; and c) a mixture of a biocompatible material and a drug, which is delivered to vascular smooth muscle cells through the needles. Herein, the mature of the biocompatible material and the drug may be attached to the end of the needles by spray or impregnation, and the drug delivery rate and period can be controlled by controlling the degradation rate or mixing ratio of the biocompatible material that is mixed with the drug.

In still another embodiment, the present invention provides a drug delivery device for attachment to the outer wall of a blood vessel, the blood delivery device comprising: a) a body made of a biocompatible material and formed so as to cover the blood vessel; and b) one or more needles made of a biocompatible material, which are connected to the inside of the body and inserted into the tunica media of the blood vessel so as to deliver a drug to vascular smooth muscle cells. The shape of the needle may be a separate type in which the body of the needle is separable from the end of the needle, or is an integral type in which the body of the needle is integral with the end of the needle. During the molding of the end of the separate-type needles or the integral-type needles, the drug may be injected so that the whole or end of the needle is composed of the mixture of the biocompatible material and the drug. In addition, the drug delivery rate and period may be controlled by controlling the degradation rate or mixing ratio of the biocompatible material that is mixed with the drug.

The above drug delivery devices enable two or more different drugs to be delivered at the same rate or different delivery rates during different periods, and the biocompatible material is preferably a biodegradable material. In addition, the body formed so as to cover the blood vessel may have a conical shape, a cylindrical shape, a pyramidal shape or polyprismal shape according to the size of the affected part or the position or kind of blood vessel, and the aspect ratio (the ratio of the height to the length of the bottom) of the needle is 1 or greater. The drug stored in the drug reservoirs may include a low-molecular-weight/high-molecular-weight drug, a peptide, a protein, RNA, sRNAi, DNA or a cell.

The present invention also provides a method for preparing a drug delivery device for attachment to the outer wall of a blood vessel, the method comprising the steps of: a) molding a body formed so as to cover a blood vessel; and b) molding one or more needles that are connected to the inside of the body and inserted into the tunica media of the blood vessel so as to deliver a drug to vascular smooth muscle cells.

During the molding of the body, one or more drug reservoirs may be formed in the body, and during the molding of the needles, microchannels serving to deliver the drug from the drug reservoirs to the tunica media of the blood vessel may be formed. In addition, the method may further comprise a step of spraying a mixture of a biocompatible material and a drug onto the end of the molded needles. The shape of the needle is a separate type in which the body of the needle is separable from the end of the needle, or is an integral type in which the body of the needle is integrally connected with the end of the needle. During the molding of the end of the separate-type needles or the integral-type needles, the drug may be injected and then dried so that the whole or end of the needle is composed of the mixture of the biocompatible material and the drug. The mixing ratio between the drug and the biocompatible is preferably 0.01-90% w/w (drug/biocompatible material).

The present invention also provides a method for delivering a drug using a body made of a biocompatible material and formed so as to cover a blood vessel, a plurality of needles connected to the body and inserted into the tunica media of the blood vessel, a plurality of drug reservoirs formed in the body, and microchannels formed in the needles and serving to deliver the drug from the drug reservoirs to the tunica media of the blood vessel, wherein the number of the needles, the size of the drug reservoirs or the structure of the microchannels is controlled to control a region into which the drug is administered, the amount of drug administered and the period of administration of the drug.

The present invention also provides a method for delivering a drug using a) a body made of a biocompatible material and formed so as to cover a blood vessel, a plurality of needles connected to the inside of the body and inserted into the tunica media of the blood vessel, and a mixture of a biocompatible material and the drug, which is delivered to vascular smooth muscle cells through the needles, or b) a body made of a biocompatible material and formed so as to cover a blood vessel, and one or more needles connected to the inside of the body and made of a biocompatible material mixed with the drug so as to deliver the drug to vascular smooth muscle cells, wherein the degradation rate of the biocompatible material or the mixing ratio between the biocompatible material and the drug is controlled to control the amount of drug administered esc the period of administration of the drug.

In the above drug delivery methods, two or more different drugs may be attached to the end of the needles and delivered at the same delivery rate or different delivery rates during different periods.

### Advantageous Effects

The inventive drug delivery device for attachment to the outer wall of blood vessels can be attached to the outer wall of a blood vessel upon laparotomy during vascular bypass surgery and can exactly deliver a desired amount of a drug to the vascular tunica media having vascular smooth muscle cells (target location) during a predetermined period using the microneedles of the device. In addition, when the drug delivery device is made of a biodegradable material, it is degraded after a specific amount of time and then absorbed in vivo, and thus a separate surgical operation for removing the device is not required.

Also, the needles can be specifically made to have the height and diameter determined according to the position of a transplanted blood vessel and cells, and the device can be fixed to the outer wall of the blood vessel through the needles so that the detachment of the device by the constriction and dilation of the blood vessel does not occur.

In addition, according to the inventive method for delivering a drug using microneedles, various drug delivery profiles can be achieved by controlling drug delivery so as to occur by diffusion through the microchannels, osmotic pressure or partitioning, or coating the ends of the microneedles with different biocompatible materials or by adding a drug to the needles during the preparation of the needles.

When the drug delivery device and drug delivery method of the present invention are used, a drug can be released into vascular cells in a spatially temporally controlled manner, and two or more different drugs can be delivered at different delivery rates during different periods, and thus the success rate of treatment can be significantly increased.

### DESCRIPTION OF DRAWINGS

FIGS. 1a and 1b are conceptual views illustrating a conventional drug delivery method that uses a gel-type structure.

FIG. 2 is a conceptual view illustrating a conventional drug delivery method that uses a cuff-type structure.

FIGS. 3 and 4 are a cross-sectional view of blood vessels and a conceptual view of a drug delivery device according to the present invention.

FIG. 5 is a conceptual view showing than a drug delivery device of the present invention is attached to blood vessels and delivered to the blood vessels.

FIGS. 6 and 7 are conceptual views of a drug delivery device that comprises microchannels and needles.

FIG. 8 is a conceptual view of drug reservoirs and microchannels.

FIGS. 9 and 10 are conceptual views of a drug delivery device having needles coated with a biocompatible material at the end.

FIG. 11 is a conceptual view of sequential delivery of multiple drugs for treatment of damaged blood vessels.

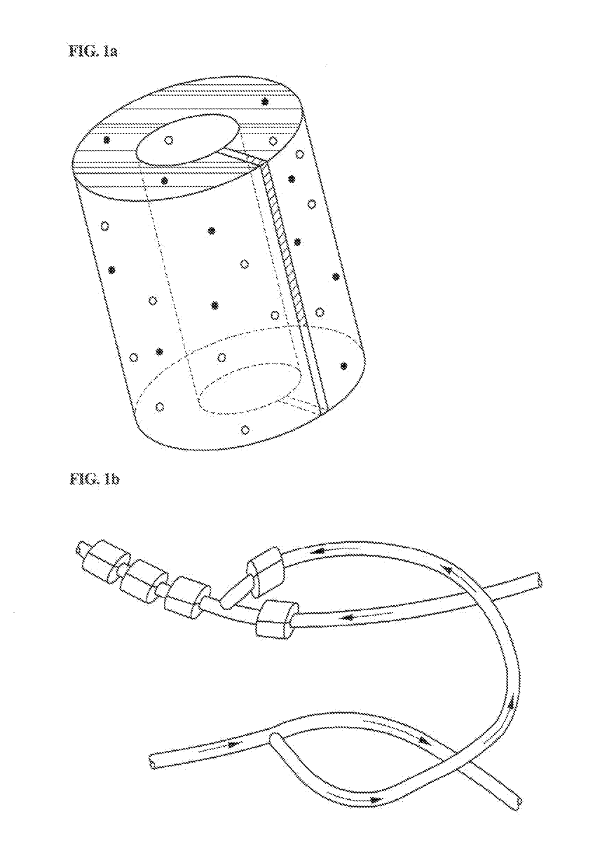

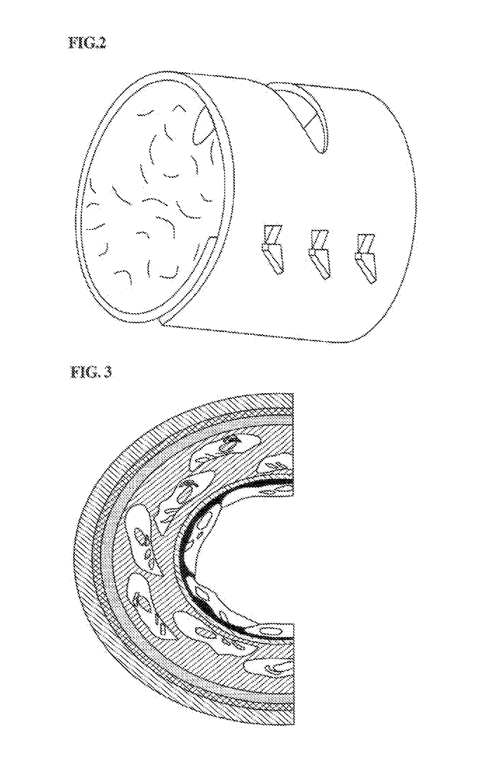

### BEST MODE

Hereinafter, preferred embodiments of the present invention will be described in detail. The terms or words used in the specifications and claims should not be limited to be construed as usual or dictionary definition but should be rather construed to be consistent with the technical spirits of the present invention.

One or important factors that cause the stenosis or occlusion of vascular grafts is the abnormal growth of vascular smooth muscle cells in vascular tissue, and many studies on drug therapy as a solution thereto have been conducted, but the constant drug delivery of drugs to vascular smooth muscle cells in the tunica media (target site) was not achieved.

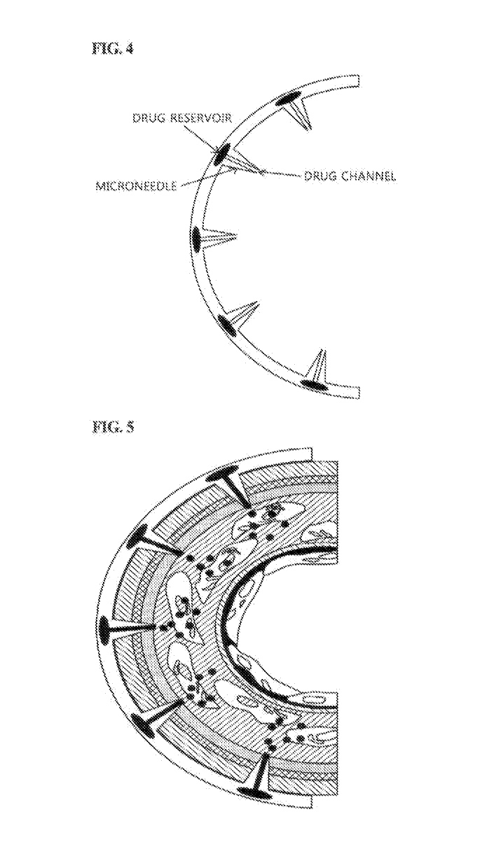

To solve this problem, according to the present invention, as can be seen in FIGS. 3, 4 and 5, the rate of delivery of a drug to a target cell is accurately controlled using a microchannel, and a microneedle is used to penetrate the outer wall of blood vessels and inject the drug directly into the vascular tunica media having vascular smooth muscle cells.

In addition, because the drug delivery device of the present invention covers the outer wall of blood vessels, it is easily attached immediately after transplantation, and because it can release a desired amount of a drug at an exact location using a microneedle, it can prevent drug loss caused by blood flow and reduce the occurrence of side effects. In addition, because the drug delivery device can be prepared using a biodegradable polymer that is degraded in the body after completion of drug delivery, a separate operation for removing the drug delivery device is not required.

The needles can be distributed outside of blood vessels in the inward direction at a constant distance and are connected with a drug reservoir by the microchannels in the device so that a drug is accurately delivered to a desired location through the channels. Also, the needles may be made of a biocompatible material so as to be degraded in the body.

The needles can be made to have the height and diameter determined according to the location of transplanted blood vessels and cells. Preferably, the aspect ratio (the ratio of height to the length of the bottom) of the needle is 1 or greater. In addition, the drug delivery device can be designed such that it is fixed to the outer wall of blood vessels through the needles so that the detachment of the device by the constriction and dilation of the blood vessel does not occur.

The total length of the drug delivery device of the present invention may vary depending on the location of the outer wall of a blood vessel and an affected part to which it is to be attached, and the number of the circumferentially arranged needles and drug reservoirs and the length and number of microchannels may vary depending on the length of the affected part, the amount of drug administered and the period of administration of the drug. Thus, the amount of drug released to the affected part can be accurately and continuously controlled according to a treatment schedule. Also, the drug is delivered only to a desired site, and thus the side effects thereof in the surrounding cells and the human body can be minimized.

In an embodiment of the present invention, the drug delivery device for attachment to the outer wall of a blood vessel comprises: a) a body made of a biocompatible material and formed to cover the blood vessel; b) one or more needles made of a biocompatible material, which are connected to the inside of the body and inserted into the tunica media of the blood vessel so as to deliver a drug to vascular smooth muscle cells; c) one or more drug reservoirs formed on the body; and d) microchannels formed in the needles and serving to deliver the drug from the drug reservoirs to the tunica media of the blood vessel.

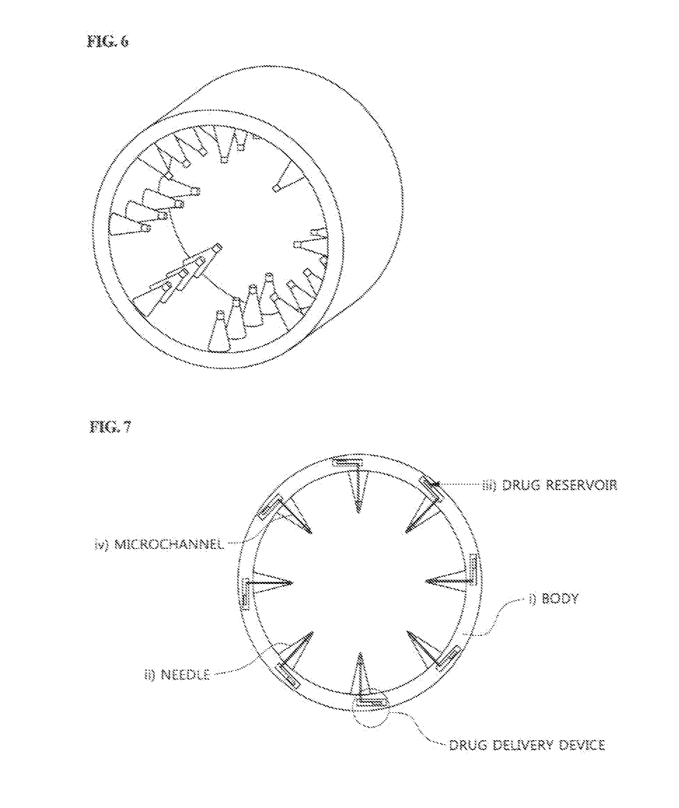

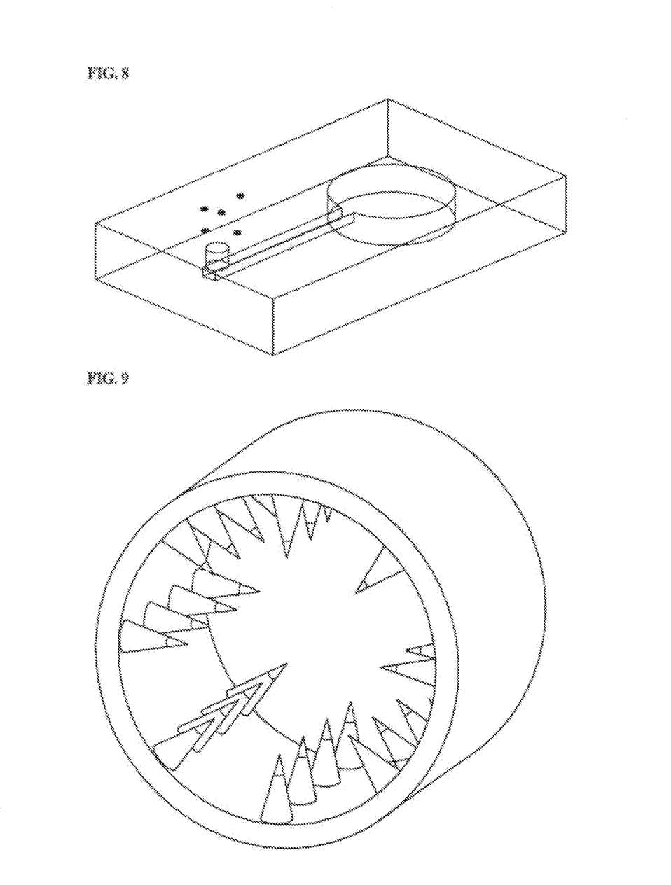

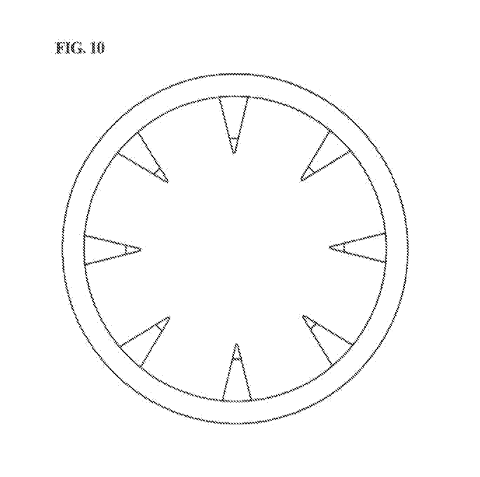

As shown in FIGS. 6 to 8, the rate and period of drug delivery by diffusion, osmotic pressure, partitioning or the like can be controlled by controlling the structure of the microchannels. The control of the drug delivery rate and period can be achieved by controlling the length, cross-sectional area and shape of the channels. A region into which the drug is administered, the amount of drug administered and the period of administration of the drug can be controlled by controlling the number of the needles, the size of the drug reservoirs and the structure of the microchannels. Preferably, two or more drugs are stored in the drug reservoirs and delivered with various delivery profiles having different delivery rates and periods.

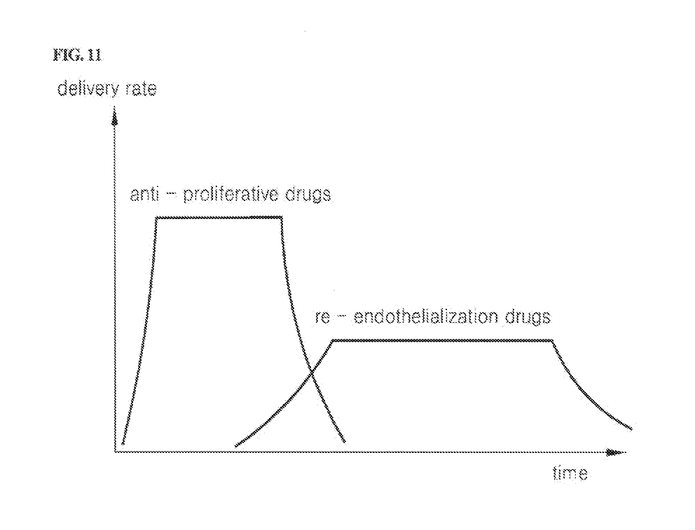

Another embodiment of the drug delivery device for attachment to the outer wall of a blood vessel is shown in FIGS. 9 and 10. As shown therein, the drug delivery device comprises: a) a body made of a biocompatible material and formed so as to cover the blood vessel; b) one or more needles made of a biocompatible material, which are connected to the inside of the body and inserted into the tunica media of the blood vessel; and c) a mixture of a biocompatible material and a drug, which is delivered to vascular smooth muscle cells through the needles.

Herein, the mixture of the biocompatible material and the drug can be attached to the end of the needles by spray or impregnation, and the drug delivery rate and period can be controlled by controlling the degradation rate and mixing ratio of the biocompatible material mixed with the drug.

Specifically, the needles (or stings) are made using the drug and the biocompatible material without a separate reservoir or channel, and the mixture of the biocompatible material (that is degraded at a predetermined time) and the drug is attached to or coated on the end of the needles, so that the drug can reach vascular smooth muscle cells in the tunica media of the blood vessel at a predetermined time and rate to prevent the stenosis and occlusion of the blood vessel.

Further, two or more different drugs may be attached to the end of the needle so that they can be delivered with various delivery profiles having the same or different delivery rates or periods.

In still another embodiment of the present invention, the drug delivery device for attachment to the outer wall of a blood vessel may comprise: a) a body made of a biocompatible material and formed so as to form cover the blood vessel; and b) one or more needles made of a biocompatible material, which are connected to the inside of the body and inserted into the tunica media of the blood vessel so as to deliver a drug to vascular smooth muscle cells.

Herein, the shape of the needle may be a separate type in which the body of the needle is separable from the end thereof, or is an integral type in which the body of the needle is integrally connected with the end thereof. During the molding of the end of the separate-type needles or the integral-type needles, a drug may be injected so that the whole or end of the needle is composed of a mixture of a biocompatible material and the drug.

In this case, the drug delivery rate and period can be controlled by controlling the degradation rate or mixing ratio of the biocompatible material mixed with the drug, and two or more different drugs can be delivered at the same delivery rate or different delivery rates during different periods.

The biocompatible material that is used in the drug delivery devices according to the above embodiments is preferably a biodegradable material. Specific examples of the biocompatible material include polyester, polyhydroxyalkanoate (PHAs), poly(α-hydroxy acid), poly(β-hydroxy acid), poly(3-hydroxybutyrate-co-valerate (PHBV), poly(3-hydroxypropionate (PHP), poly(3-hydrocyhexanoate (PHH), poly(4-hydroxy acid), poly(4-hydroxybutyrate), poly(4-hydroxyvalerate), poly(4-hydroxyhexanoate), poly(esteramide), polycaprolactone, polylactide (PLA), polyglycolide (PGA), poly(lactide-co-glycolide (PLGA), polydioxanone, polyorthoester, polyanhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine acrylate), polyalkylene oxalate, polyphosphazene, PHA-PEG, polyvinyl pyrrolidone, polybutadiene, polyhydroxybutyric acid, polymethyl methacrylate, polymethacrylic acid ester, polypropylene, polystyrene, polyvinyl acetal diethylamino acetate, polyvinyl acetate, polyvinyl alcohol, polyvinyl butyral, polyvinyl formal, a vinyl chloride-propylene-vinyl acetate copolymer, a vinyl chloride-vinyl acetate copolymer, a cumaroneindene polymer, dibutylaminohydroxypropyl ether, an ethylene-vinylacetate copolymer, glycerol distearate, a 2-methyl-5-vinylpyridine methacrylate-methacrylic acid copolymer, hyaluronic acid, myristic acid, palmitic acid, stearic acid, behenic acid, cellulose, monosaccharide, biose, triose, oligosaccharide, polysaccharide, maltose, lactose, sucrose, oligosaccharides, polysaccharides, dextrane, glucomannan, glucosamine, chitosan, heparin, alginate, inulin, starch, glycogen, chitin, chondroitin, dextrin, keratan sulfate, beef tallow, whale wax, beeswax, paraffin wax, cantor wax, and polymer materials such as polyethylene (UHMWPE), polyetrafluoroethylene, polysulfone, polyethylene terephthalate, polyformaldehyde, polyamides, polyolefins, polytetrafloroethylene, polyfluorocarbons, polyvinyl chloride, polyacrylonitrile, hydrogels, polyurethanes, silicones, ethylene vinyl acetate, thermoplastic elastomeric polymers, acrylics, perfloroether copolymers, poly 2-hydroxy esters, nylon, and silicon rubber.

In addition, examples or the biocompatible material that may be used in the present invention include metals or alloys thereof, such as cobalt (Co), titanium (Ti), stainless steel, Teflon, zinc (Zr), chromium (Cr), nickel (Ni), copper (Cu), silver (Ag), gold (Au), aluminum (Al), Ni—Ti, molybdenum (Mo), tantalum (Ta), platinum (Pt), amalgam, and human enamel, and ceramic materials such as alumina (Al₂O₃), zirconia (ZrO₂), carbon, bioglass, hydroxyapatite (HA), calcium aluminate, tricalcium phosphate, calcium sulfate, calcium phosphate, human dentin, and cortical bone.

In addition, when two or more different drugs are delivered with different delivery profiles using the drug delivery devices of the above various embodiments, as shown in FIG. 11, an anti-proliferative drug for inhibiting the abnormal growth of vascular smooth muscle cells is first delivered for one day to 2 weeks, and then a re-endothelialization drug for stimulating the growth of vascular endothelial cells is delivered for one week to one month. In this case, the limitations of conventional drug delivery methods can be overcome.

Moreover, the body formed so as to cover the blood vessel may have a conical shape, a cylindrical shape, a pyramidal shape or polyprismal shape according to the size of the affected part or the position or kind of blood vessel. The drug stored in the drug reservoirs may include various drugs such as low-molecular-weight/high-molecular-weight drugs, peptides, proteins, RNA, sRNAi, DNA or cells.

Meanwhile, the above-described drug delivery device can be prepared by a method comprising the steps of: a) molding a body formed so as to cover a blood vessel; and b) molding one or more needles that are connected to the inside of the body and inserted into the tunica media of the blood vessel so as to deliver a drug to vascular smooth muscle cells.

During the molding of the body, one or more drug reservoirs can be formed in the body, and during the molding of the needles, microchannels serving to deliver the drug from the drug reservoirs to the tunica media of the blood vessel can be formed. In addition, the method may further comprise a step of spraying a mixture of a biocompatible material and a drug onto the end of the molded needles.

Also, the shape of the needle may be a separate type in which the body of the needle is separable from the end of the needle, or may be an integral type in which the body of the needle is integrally connected with the end of the needle. During the molding of the separate-type needles or the integral-type needles, the drug may be injected and then dried so that the whole or end of the needle is composed of the mixture of the biocompatible material and the drug. The mixing ratio between the drug and the biocompatible is preferably 0.01-90% w/w (drug/biocompatible material).

As described above, according to the present invention, a design of a micro-drug delivery device that could not be achieved to date can be achieved by selecting a microstructure for drug delivery or the kind and mixing ratio of biocompatible material and drug. In addition, when the drug delivery device is prepared using a biodegradable material, it is degraded naturally after completion of drug delivery or is attached so that it does not affect the body, and thus it does not need to be removed by re-surgery and can be easily used for treatment.

Although the preferred embodiments of the present invention have been disclosed for illustrative purposes, those skilled in the art will appreciate that various modifications, additions and substitutions sure possible, without departing from the scope and spirit or the invention as disclosed in the accompanying claims.

1. A drug delivery device for attachment to the outer wall of a blood vessel, the blood delivery device comprising: a) a body made of a biocompatible material and formed so as to cover the blood vessel; b) one or more needles made of a biocompatible material, which are connected to the inside of the body and inserted into the tunica media of the blood vessel so as to deliver a drug to vascular smooth muscle cells; c) one or more drug reservoirs formed in the body; and d) microchannels formed in the needles and serving to deliver the drug from the drug reservoirs to the tunica media of the blood vessel.

2. The drug delivery device of claim 1, wherein the rate and period of drug delivery by diffusion, osmotic pressure, and partitioning are controlled by controlling the structure of the microchannels.

3. The drug delivery device of claim 2, wherein the control of the drug delivery rate and period are achieved by controlling the length, cross-sectional area or shape of the channels.

4. The drug delivery device of any one of claims 1 to 3, wherein the drug delivery device is configured to deliver two or more different drugs at the same delivery rate or different delivery rates during different periods.

5. The drug delivery device of any one of claims 1 to 3, wherein the biocompatible material is a biodegradable material.

6. The drug delivery device of any one of claims 1 to 3, wherein the body formed so as to cover the blood vessel has a conical shape, a cylindrical shape, a pyramidal shape or polyprismal shape according to the size of an affected part or the position or kind of blood vessel.

7. The drug delivery device of any one of claims 1 to 3, wherein the aspect ratio (the ratio of the height to the length of the bottom) of the needle is 1 or greater.

8. The drug delivery device of any one of claims 1 to 3, wherein the drug stored in the drug reservoirs includes a low-molecular-weight/high-molecular-weight drug, a peptide, a protein, RNA, sRNAi, DNA or a cell.

9. A drug delivery device for attachment to the outer wall of a blood vessel, the blood delivery device comprising: a) a body made of a biocompatible material and formed so as to cover the blood vessel; b) one or more needles made of a biocompatible material, which are connected to the inside of the body and inserted into the tunica media of the blood vessel; and c) a mixture of a biocompatible material and a drug, which is delivered to vascular smooth muscle cells through the needles.

10. The drug delivery device of claim 9, wherein the mixture of the biocompatible material and the drug is attached to the end of the needles by spray or coating.

11. The drug delivery device of claim 9 or 10, wherein the drug delivery rate and period are controlled by controlling the degradation rate or mixing ratio of the biocompatible material that is mixed with the drug.

12. The drug delivery device of claim 9 or 10, wherein the drug delivery device is configured to deliver two or more different drugs at the same delivery rate or different delivery rates during different periods.

13. The drug delivery device of claim 9 or 10, wherein the biocompatible material is a biodegradable material.

14. The drug delivery device of claim 9 or 10, wherein the body formed so as to cover the blood vessel has a conical shape, a cylindrical shape, a pyramidal shape or polyprismal shape according to the size of an affected part or the position or kind of blood vessel.

15. The drug delivery device of claim 9 or 10, wherein the aspect ratio (the ratio of the height to the length of the bottom) of the needle is 1 or greater.

16. The drug delivery device of claim 9 or 10, wherein the drug stored in the drug reservoirs includes a low-molecular-weight/high-molecular-weight drug, a peptide, a protein, RNA, sRNAi, DNA or a cell.

17. A drug delivery device for attachment to the outer wall of a blood vessel, the blood delivery device comprising: a) a body made of a biocompatible material and formed so as to cover the blood vessel; and b) one or more needles made of a biocompatible material, which are connected to the inside of the body and inserted into the tunica media of the blood vessel so as to deliver a drug to vascular smooth muscle cells.

18. The drug delivery device of claim 17, wherein the shape of the needle is a separate type in which the body of the needle is separable from the end of the needle, or is an integral type in which the body of the needle is integrally connected with the end of the needle.

19. The drug delivery device of claim 17, wherein the drug is included during molding of the end of the separate-type needles or the integral-type needles so that the whole or end of the needle is composed of the mixture of the biocompatible material and the drug.

20. The drug delivery device of any one of claims 17 to 19, wherein the drug delivery rate and period are controlled by controlling the degradation rate or mixing ratio of the biocompatible material that is mixed with the drug.

21. The drug delivery device of any one of claims 17 to 19, wherein the drug delivery device is configured to deliver two or more different drugs at the same delivery rate or different delivery rates during different periods.

22. The drug delivery device of any one of claims 17 to 19, wherein the biocompatible material is a biodegradable material.

23. The drug delivery device of any one of claims 17 to 19, wherein the body formed so as to cover the blood vessel has a conical shape, a cylindrical shape, a pyramidal shape or polyprismal shape according to the size of an affected part or the position or kind of blood vessel.

24. The drug delivery device of any one of claims 17 to 19, wherein the aspect ratio (the ratio of the height to the length of the bottom) of the needle is 1 or greater.

25. The drug delivery device of any one of claims 17 to 19, wherein the drug stored in the drug reservoirs includes a low-molecular-weight/high-molecular-weight drug, a peptide, a protein, RNA, sRNAi, DNA or a cell.

26. A method for preparing a drug delivery device for attachment to the outer wall of a blood vessel, the method comprising the steps of: a) fabricating a body formed so as to cover a blood vessel; and b) fabricating one or more needles that are connected to the inside of the body and inserted into the tunica media of the blood vessel so as to deliver a drug to vascular smooth muscle cells.

27. The method of claim 26, wherein microchannels serving to deliver the drug from the drug reservoirs to the tunica media of the blood vessel are formed during fabrication of the needles.

28. The method of claim 26, further comprising a step of coating a mixture of a biocompatible material and the drug onto the end of the molded needles.

29. The method of claim 26, wherein the shape of the needle is a separate type in which the body of the needle is separable from the end of the needle, or is an integral type in which the body of the needle is integrally connected with the end of the needle.

30. The method of claim 29, wherein the drug is included during fabrication of the end of the separate-type needles or the integral-type needles and then dried so that the whole or end of the needle is composed of a mixture of the biocompatible material and the drug.

31. The method of claim 30, wherein the mixing ratio between the drug and the biocompatible is 0.01-90% w/w (drug/biocompatible material).

32. A method for delivering a drug using a body made of a biocompatible material and formed so as to cover a blood vessel, a plurality of needles connected to the body and inserted into the tunica media of the blood vessel, a plurality of drug reservoirs formed in the body, and microchannels formed in the needles and serving to deliver the drug from the drug reservoirs to the tunica media of the blood vessel, wherein the number of the needles, the size of the drug reservoirs or the structure of the microchannels is controlled to control a region into which the drug is administered, the amount of drug administered and the period of administration of the drug.

33. The method of claim 32, wherein two or more different drugs are stored in the drug reservoirs and delivered at the same delivery rate or different delivery rates during different periods.

34. A method for delivering a drug using a) a body made of a biocompatible material and formed so as to cover a blood vessel, a plurality of needles connected to the inside of the body and inserted into the tunica media of the blood vessel, and a mixture of a biocompatible material and the drug, which is delivered to vascular smooth muscle cells through the needles, or b) a body made of a biocompatible material and formed so as to cover a blood vessel, and one or more needles connected to the inside of the body and made of a biocompatible material mixed with the drug so as to deliver the drug to vascular smooth muscle cells, wherein the degradation rate of the biocompatible material or the mixing ratio between the biocompatible material and the drug is controlled to control the amount of drug administered and the period of administration of the drug.

35. The method of claim 34, wherein two or more different drugs are attached to the end of the needles and delivered at the same delivery rate or different delivery rates during different periods.
